(19) United States
(12) Patent Application Publication
(10) Pub. No.: US 2022/0015327 A1
(43) Pub. Date: Jan. 20, 2022

(54) Temperature control of a climatic zone of an insect-breeding facility

(71) Applicant: YNSECT, Évry-Courcouronnes Cedex (FR)

(72) Inventors: Loic CLESSE, ROISIEL (FR); Thibault DU JONCHAY, CHEVRIÈRES (FR); Arturo ESCAROZ CETINA, EINDHOVEN (NL); François SALA, LYON (FR); Cyrille CANITROT, DIJON (FR); Fabrice BERRO, PARIS (FR)

(73) Assignee: YNSECT, Évry-Courcouronnes Cedex (FR)

(21) Appl. No.: 17/294,435

(22) PCT Filed: Nov. 19, 2019
(86) PCT No.: PCT/FR2019/052756

§ 371 Date: May 17, 2021

(30) Foreign Application Priority Data
Nov. 20, 2018 (FR) 1871610

(51) Int. Cl. A01K 1/00; A01K 67/033

(52) U.S. Cl. CPC A01K 1/0064; A01K 67/033

(57) ABSTRACT

The invention concerns an insect-breeding facility comprising a climatic zone (Z1, Z2) for storing the insects. The facility comprises an air-conditioning zone (Z4) comprising an air-conditioning system, for bringing air to a first temperature, and simultaneously for bringing air to a second temperature (T2). A first set of pipes (C1) is configured to transport the air at the first temperature from the air-conditioning zone (Z4) to the climatic zone (Z1, Z2) and to deliver it there, a second set of pipes (C2) is configured to transport the air at the second temperature from the air-conditioning zone (Z4) to the climatic zone (Z1, Z2) and to deliver it there. The facility also comprises a device for extracting air from the climatic zone (Z1, Z2). The invention also concerns a corresponding method for air conditioning in a climatic zone (Z1, Z2) of an insect-breeding facility.

The present invention concerns the field of rearing insects.

The insects concerned by the invention are for example the Coleoptera, Diptera, Lepidoptera, Isoptera, Orthoptera, Hymenoptera, Blattoptera, Hemiptera, Heteroptera, Ephemeroptera and Mecoptera, preferably, Coleoptera, Diptera, Orthoptera, Lepidoptera.

The term “insect” is employed to designate any stage of development of the egg or egg pod to the adult insect, and the invention is more particularly directed to the rearing of insects from the larval stage to the adult insect.

Insect farming is experiencing something of a boom. The production of insects has many attractions, whether for the agro-industry, as certain species of edible insects are rich in proteins, or in other industrial fields. Typically, the exoskeleton of insects is constituted in large part by chitin, a known derivative of which is chitosan. The applications of chitin and/or chitosan are many: cosmetics (cosmetic composition), medical and pharmaceutical (pharmaceutical composition, treatment of burns, biomaterials, corneal dressings, surgical sutures), dietetic and dietary, technical (filtering agent, texturing agent, flocculating agent or adsorbant, in particular for water filtration or pollution control), etc. In fact, chitin and/or chitosan are biocompatible, biodegradable and non-toxic materials.

Document FR3034622 presents a farm adapted for the rearing of insects at an industrial scale. The rearing implements rearing containers (typically tubs) which are stackable, in one or more columns, to form basic rearing units. The basic rearing units are stored, and, when a rearing operation is to be carried out, the containers are brought to a station configured for carrying out the operation, grouped into basic rearing units or ungrouped singly.

The insects thus live in a zone in which they grow and develop between the rearing operations. It is thus important in this zone to maintain environmental conditions favorable to their health, their well-being and their fast growth.

Environmental conditions in particular refers to the temperature of the air, the hygrometry, and the level of carbon dioxide (CO₂) present in the air.

Document CN107372375 indicates in general terms the importance of controlling the temperature, the humidity and the level of CO₂ in a silk worm farm. This document describes a farming facility comprising a sensor for temperature, humidity in the air, and CO₂.

In the context of rearing insects at industrial scale, there is not however known in the state of the art any device making it possible to obtain and maintain properly controlled and homogenous environmental conditions. For example, as regards the control of the temperature in the rearing farm, two main issues arise in rearing at very large scale. One issue is that the very large amount of insects (typically several tens of tonnes of insects in a rearing farm) generates a very high amount of heat. Moreover, it is difficult to ensure sufficient homogeneity of the temperature.

However, the optimum temperature range for the growth of insects is generally rather restricted. As regards the Tenebrio molitor mealworm, for example, although it is active between 15° C. and 40° C. and can survive at a slightly lower or slightly higher temperature, the rate of growth of this species is a maximum at a temperature of around 25° C. Similarly, in zones of the farm where what is sought is not maximum growth of the insects, but for example laying, a rather precise temperature must be maintained.

Obtaining such a temperature in a rearing zone of large size, relatively homogenously, and maintaining it despite possible temporal and spatial variations, is an issue that is unknown and thus a fortiori unsolved in the state of the art.

The same applies for the humidity level in the air. As a matter of fact, although quite a broad range of relative humidity is tolerated, a humidity that is too low can slow the growth of that insect and a humidity that is too high can promote the development of fungal diseases.

Thus, the invention provides an insect rearing farm comprising a climate zone of which the environmental conditions, in particular as regards the temperature, are controlled by an air conditioning system configured for farming on a large scale.

Thus, the invention relates to an insect rearing farm comprising a climate zone which comprises a set of racks for the storage of the insects in rearing containers and an air conditioning zone comprising an air conditioning system configured for regulating the air to a first temperature. The farm comprises a first set of ducts configured to transport the air at the first temperature from the air conditioning zone to the climate zone and to deliver said air at the first temperature into said climate zone. The air conditioning system is furthermore configured for regulating the air temperature to a second temperature conjointly with the temperature regulation of air to the first temperature. The farm also comprises a second set of ducts configured to transport the air at the second temperature from the air conditioning zone to the climate zone and to deliver said air at the second temperature into said climate zone. The air at the first temperature and the air at the second temperature mix in said climate zone.

A provision of air at two different temperatures in the farm enables effective and fast control of its ambient temperature. Furthermore, in a farm equipped according to the invention, it is possible to generate a stream of air enabling proper renewal of the air but also good homogeneity of the temperatures in the climate zone. Lastly, the provision of air in accordance with two different regimes enables an optimization of the energy need for cooling the rearing farm.

According to certain embodiments, the device for extracting air comprises a third set of ducts that are configured for the return of air, from the climate zone, into the air conditioning zone.

The extraction of air from the climate zone considered may be carried out in part via the third set of ducts, which enables the recycling of a portion of the air coming from the farm, and its cooling in the air conditioning zone for it to be returned to the farm (via the first set of ducts and/or the second set of ducts). The portion of the air which is not extracted by the third set of ducts may be extracted by suitable air extractors, to the atmosphere outside the farm. Extraction to the outside of the farm enables renewal of the air and proves advantageous when the outside air is at a lower temperature than the temperature aimed for in the climate zone (which enables cooling of the climate zone without requiring energy to be consumed in obtaining fresh air, such that this can be said to be “freecooling”.

The first set of ducts may comprise a plurality of flues for distributing air at the first temperature, each formed from a longitudinal duct comprising air ejection nozzles distributed along said flue for distributing air at the first temperature, and in which the second set of ducts may comprise a plurality of flues for distributing air at the second temperature, each formed from a duct comprising air ejection nozzles distributed along said flue for distributing air at the second temperature.

The racks of the climate zone may be organized on opposite sides of parallel aisles and one aisle in every two is then a handling aisle configured for the passage of the rearing containers in the climate zone as well as for the entry of the rearing containers into the climate zone and their exit from the climate zone, and one aisle in every two is a ventilation aisle which comprises a succession, in a predefined sequence, of flues for distributing air at the first temperature and of flues for distributing air at the second temperature, said flues for distributing air at the first temperature and at the second temperature extending substantially vertically between the racks.

The ventilation aisles may further comprise air extraction flues of the air extraction device, said air extraction flues extending substantially vertically between the racks.

The flues for distributing and extracting air may be arranged in each ventilation aisle according to the following single or several times repeated sequence: air extraction flue, flue for distributing air at the first temperature, flue for distributing air at the second temperature, flue for distributing air at the first temperature, flue for distributing air at the second temperature, flue for distributing air at the first temperature, air extraction flue.

Alternatively, the air distribution flues are arranged in each ventilation aisle in the following single or several times repeated sequence: flue for distributing air at the first temperature, flue for distributing air at the second temperature, flue for distributing air at the first temperature, flue for distributing air at the second temperature.

In an insect rearing farm comprising an air extraction device, the farm may further comprise air return vents of the air extraction device, located at an end of said aisles.

The insect rearing farm may further comprise air extractors configured to extract air from the climate zone to the outside of the farm.

The air extractors may be in the upper part of the climate zone, on towers juxtaposed against a wall of the farm.

The flues for distributing air at the first temperature and the flues for distributing air at the second temperature may be disposed above the racks.

The racks may then be organized into one or more strata each comprising, in a same horizontal plane, several parallel rows, in which a flue for distributing air at the first temperature and a flue for distributing air at the second temperature are disposed above each row, and an air extraction flue of the air extraction device is disposed under each rack. For example, two successive strata may be separated by a thermally insulating floor.

The racks of the climatic zone may be organized, in one or more strata, on opposite sides of parallel aisles configured for the passage of the rearing containers in the climate zone as well as for the entry of the rearing containers into the climate zone and their exit from the climate zone and in which above each rack there extend a flue for distributing air at the first temperature and a flue for distributing air at the second temperature, and above each aisle there extends a flue for distributing air at the first temperature.

The racks may be organized into groups of racks, each rack group being formed from an aisle and from racks located directly on opposite sides of that aisle, it being possible for each group of racks to be separated from adjacent groups of racks by thermally insulating walls.

The racks of the climate zone may be organized on opposite sides of parallel aisles and one aisle in every two is then a handling aisle configured for the passage of the rearing containers in the climate zone as well as for the entry of the rearing containers into the climate zone and their exit from the climate zone, and one aisle in every two is a ventilation aisle above which extends a flue for distributing air at the first temperature of which the air ejection nozzles are oriented towards a ground of the farm.

The air ejection nozzles of the flues for distributing air at the second temperature T2 may be oriented towards a flue for distributing air at the first temperature.

In any embodiment, free spaces may advantageously be configured between the air distribution flues and the racks to enable homogenization of the temperature.

Walls may be provided facing the air ejection nozzles of the air distribution flues, so as to promote the mixing of the air introduced into the climate zone respectively at the first temperature and at the second temperature.

The air conditioning system may furthermore enable the control of the humidity level of the air at the first temperature and/or of the air at the second temperature.

The climate zone may furthermore comprise at least one water mister.

The first temperature may be greater than the second temperature, the air conditioning system being configured to produce two to four times more air at the first temperature than air at the second temperature.

The first set of ducts and the air extraction device may generate the majority of the stream of air into the climate zone, and the second duct set enables temperature correction.

The first set of ducts and the second set of ducts may each comprise branches provided with control valves making it possible to modulate the rate of flow of air into each of said branches.

In an insect rearing farm comprising several distinct climate zones, the first set of ducts and the second set of ducts may comprise at least one distinct branch per climate zone.

According to another aspect, the invention relates to a method of air conditioning in a climate zone of an insect rearing farm, the method comprising introducing air at a first temperature into the climate zone conjointly with introducing air at a second temperature into the climate zone and with extracting from said climate zone a similar amount of air to the amount of air introduced, and controlling the amount of air respectively introduced at the first temperature and at the second temperature according to the difference between a temperature setpoint and a temperature measured at one or more points of the climate zone. In such a method, the first temperature may be greater than the second temperature, and the first temperature and the second temperature may both be less than the setpoint temperature.

Still other particularities and advantages of the invention will appear in the following description.

In the accompanying drawings, given by way of non-limiting example:

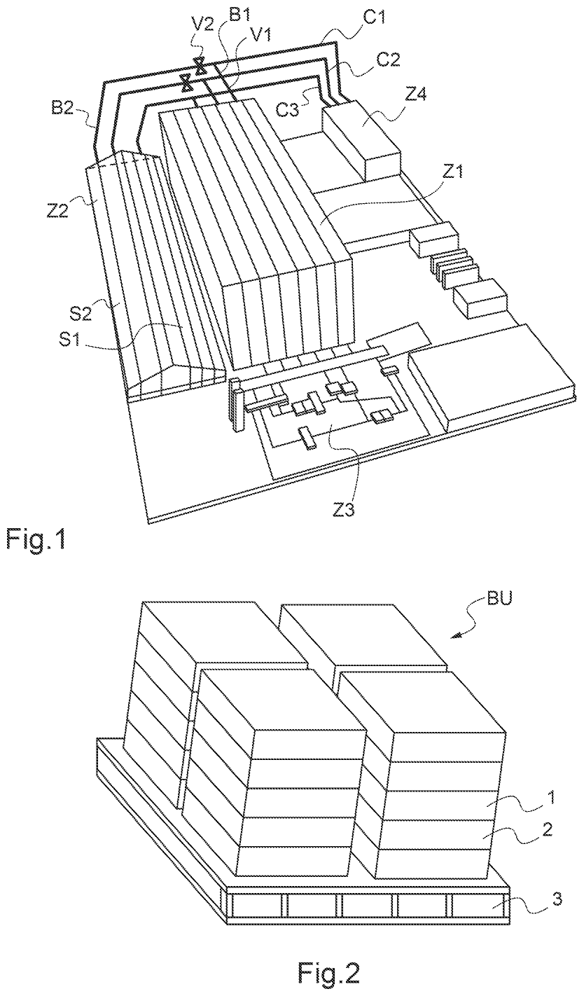

FIG. 1 shows a diagrammatic view in three dimensions of an example of general organization of an insect rearing farm in accordance with an embodiment of the invention;

FIG. 2 shows a diagrammatic view in three dimensions of a set of rearing containers which can be used in an insect farm;

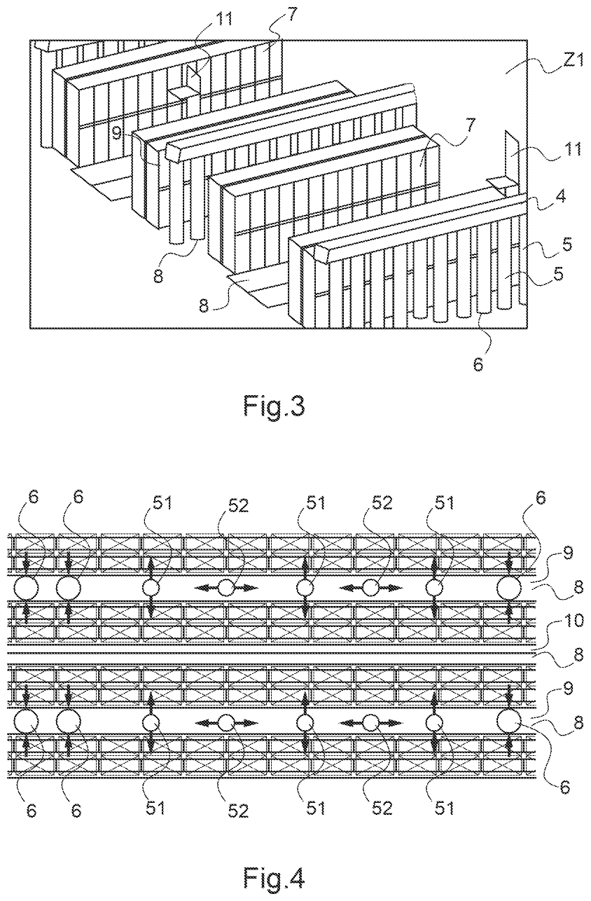

FIG. 3 shows, in a concept diagram, a first example of general configuration of a climate zone of an insect rearing farm in accordance with an embodiment of the invention;

FIG. 4 shows, in a diagrammatic plan view, a climate zone according to the configuration of FIG. 3; according to a first embodiment;

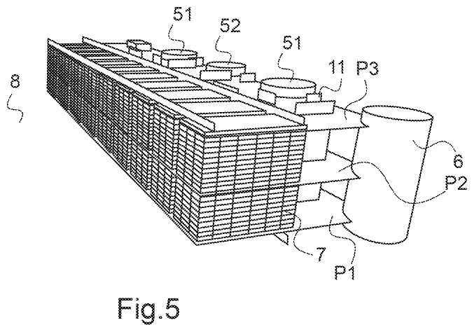

FIG. 5 shows, in a diagrammatic view in three dimensions, a climate zone according to the configuration of FIGS. 3 and 4;

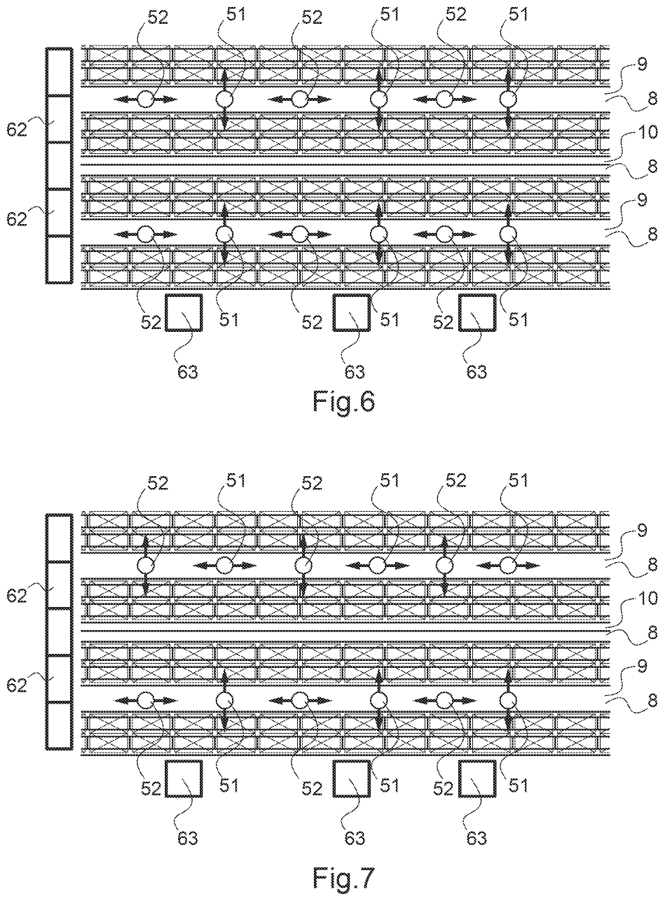

FIG. 6 shows, in a diagrammatic plan view, a climate zone according to the configuration of FIG. 3; according to a second embodiment;

FIG. 7 shows a diagrammatic plan view of a variant of the climate zone of FIG. 6;

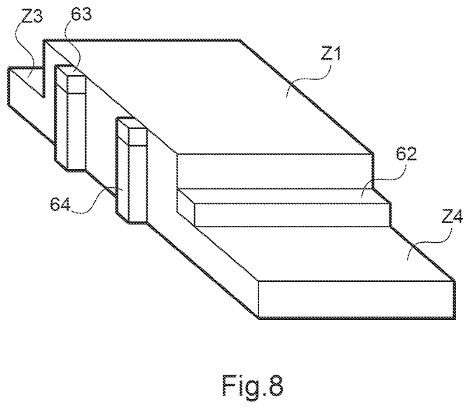

FIG. 8 shows a diagrammatic view in three dimensions of an example of general organization of an insect rearing farm in accordance with an embodiment of the invention;

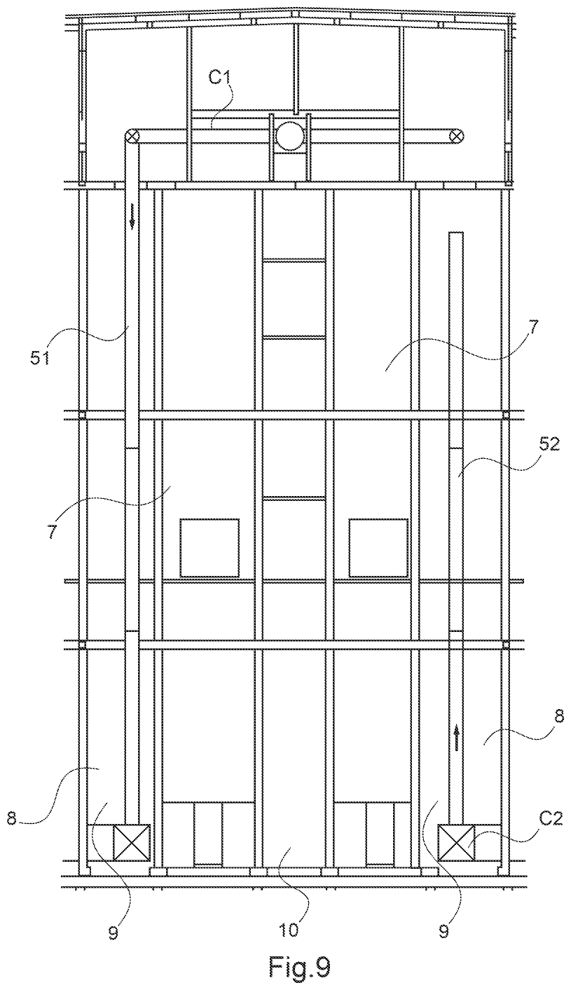

FIG. 9 shows a two-dimensional view of a configuration example of the first set and of the second set of ducts in a climate zone of a insect rearing farm according to the invention;

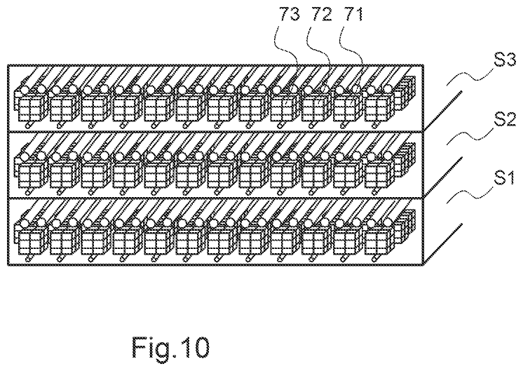

FIG. 10 shows a diagrammatic view in three dimensions of a second general configuration example of a climate zone of an insect rearing farm in accordance with an embodiment of the invention;

FIG. 11 shows a diagrammatic view of the configuration of FIG. 10 on a two-dimensional plane;

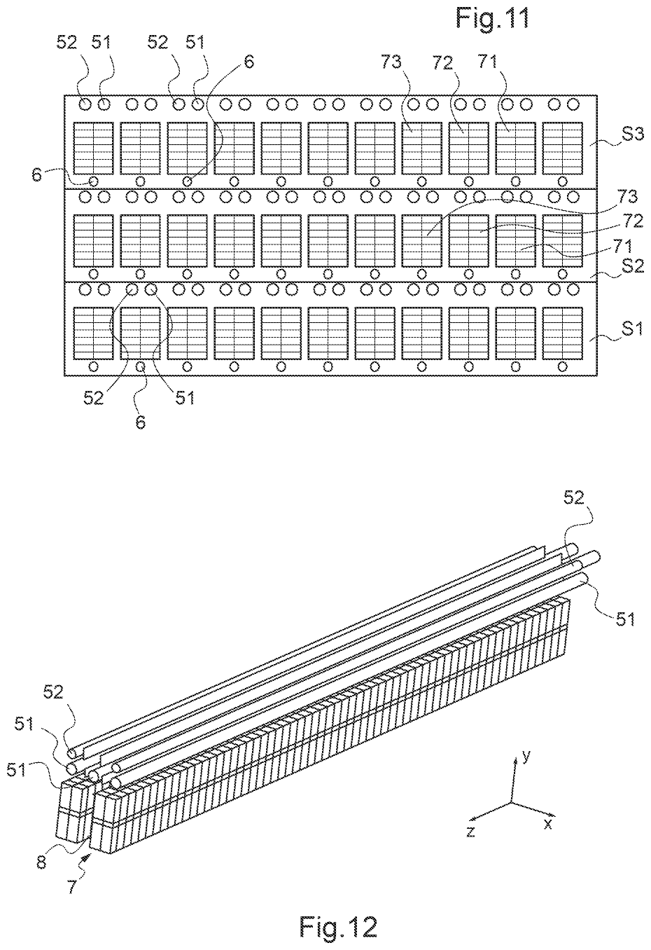

FIG. 12 shows, in a partial view in three dimensions, a variant of the configuration of FIGS. 10 and 11;

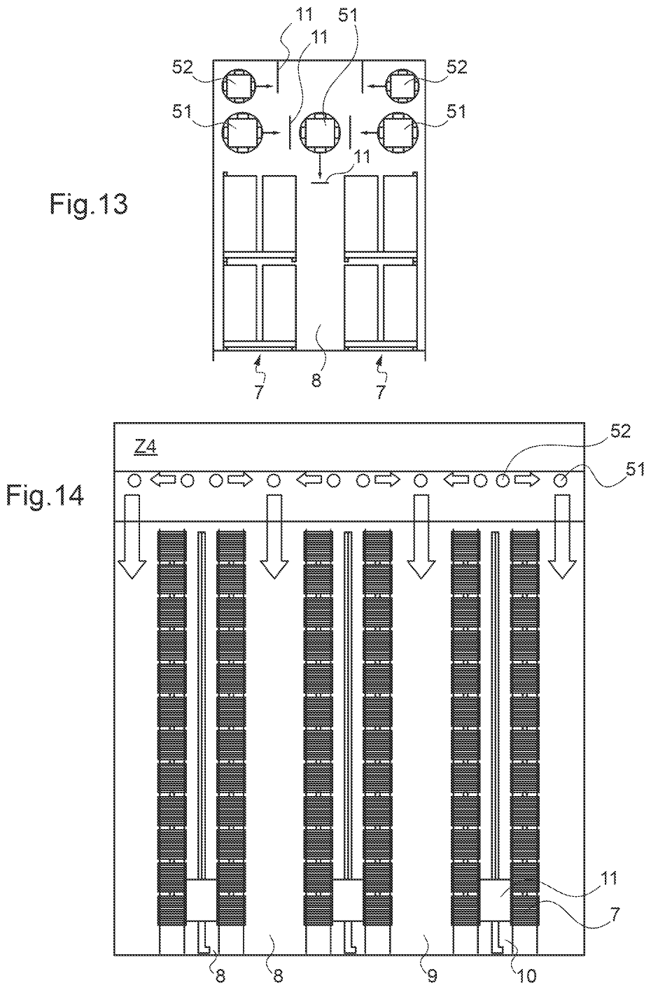

FIG. 13 shows the variant of FIG. 12 on a two-dimensional plane;

FIG. 14 shows, on a two-dimensional plane, a third example of general configuration of a climate zone of an insect rearing farm in accordance with an embodiment of the invention;

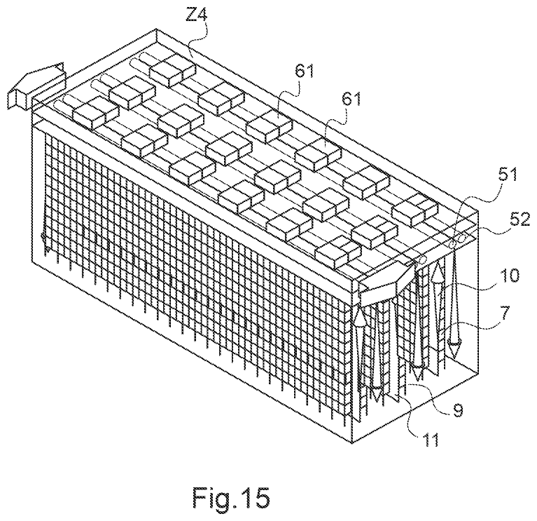

FIG. 15 shows a diagrammatic view in three dimensions of a variant of the climate zone of FIG. 14.

FIG. 1 shows an insect rearing farm, here represented in the form of a diagrammatic view in three dimensions.

The rearing of insects can in particular be envisioned as an organized facility enabling the laying of eggs by adult insects for the production of larvae, some larvae being reared to reach the adult stage for the laying of new eggs, the adults being renewed regularly (for example further to their death) by young adults ensuring new laying and so forth. The final product of the production can be eggs, and/or larvae, and/or nymphs, and/or adult insects.

The farm represented by way of example comprises a first climate zone Z1 organized for the storage of the insects during their growth.

In this first climate zone Z1, the insects increase in size under controlled, directed and optimized environmental conditions (defined by environmental parameters including temperature, hygrometry, etc.).

As mentioned above, the concept of rearing insects comprises the growth of adult insects up to a desired stage, but may also comprise all the phases preceding obtaining an adult insect (or imago), from the laying of the eggs (or egg pod) and passing via their hatching, the larval stage, any nymph stage, pupa (all the intermediate stages), etc. Thus, the farm represented comprises a second climate zone Z2, which is dedicated to the reproduction and the laying of the insects. The reproduction and laying zone could alternatively be provided in a part or silo of the first climate zone Z1.

Although the farm serving as example in the present invention comprises two climate zones Z1, Z2, the farm of the invention may of course comprise a single climate zone, or more than two climate zones.

The farm represented here also comprises a third zone Z3, organized for carrying out of one or more rearing sequences or operations. The conduct of the rearing comprises the implementation of a succession of rearing operations or sequences. A sequence or “operational sequence” comprises one or more successive predefined operations, and is carried out between two phases of growth (except when this is the sending of the insects to another process).

The rearing operations correspond to operations that must be conducted in order to maintain life, good growth and/or the optimization of the insect rearing conditions.

The third zone Z2 comprises in particular one or more specialized work stations P1, P2 for carrying out one or more rearing operations.

The insects (eggs, larvae, nymphs, or adults) are reared in containers, which may be grouped into sets called basic rearing units. In growth phases, the containers are stored in the first climate zone Z1, for example in racks for pallets.

An example of a basic rearing unit is shown in FIG. 2 in a representation of principal in three dimensions. In order to facilitate the handling thereof, each basic rearing unit may be carried by a pallet, as shown in FIG. 2.

In particular, the rearing containers 1, 2 can be stackable crates or tubs. By stackable tubs or crates is meant in particular tubs or crates that are superimposed on one another in a slightly nested manner, which achieves a certain stability for the column of crates thus formed.

As shown in FIG. 2, the containers 1, 2 are palletized, i.e. grouped together into basic units BU on a loading pallet 3. The pallet 3 may particularly, but not exclusively, be a pallet of conventional size, i.e. typically a “pallet Europe” type of pallet, or a half-pallet of that type.

By way of example, a basic rearing unit BU can typically group together eight to one hundred containers, and comprise one, two, three, or four piles of containers, or even more. The height of an entire basic rearing unit may for example be comprised between 160 and 230 cm, and typically of the order of 200 cm.

In what are referred to as growth phases, each basic unit may be stored in a part of the first climate zone Z1 called silo, and which has environmental conditions that are optimized for the stage of development (or maturity) of the insects of the basic unit considered.

The silos are isolated from each other by a suitable partition. This partitioning of silos can utilize air curtains, or any other partitioning means, in particular physical partitions, making it possible to separate two zones in order to be able to ensure therein two different atmospheric conditions (temperature, hygrometry, etc.) and sanitary separation between the silos. The first climate zone Z1 may comprise several distinct silos.

The silos so constituted may be dedicated to different stages of maturity of the insects, or to several rearing processes, in accordance with embodiments of the invention and which are conducted in parallel in a farm.

For example, the conduct of the rearing may comprise several cycles with which may be associated different rearing conditions, that is to say different optimum environmental parameters. Typically, the rearing may comprise:
an incubation cycle for the production of juveniles by fertile adults, this cycle being carried out at a temperature and in conditions of humidity that are relatively high.
a cycle of reproduction, from the juvenile to the fertile mature young adult passing via nymphosis in suitable environmental conditions;
a production cycle (or “fattening”) from the juvenile to the mature larva for killing, with a lower temperature and humidity than for the cycles cited above.
   In the example of an insect rearing farm represented here, the insects are stored in the first climate zone Z1 at the time of the production cycle. The reproduction cycle is conducted in a first silo S1 of the second climate zone Z2. The incubation cycle is conducted in a second silo S2 of the second climate zone Z2.
   Below in the present description, the term climate zone will be employed both for a climate zone as such, but also for a silo of a climate zone, since a silo may be considered as a distinct zone in which particular environmental conditions must be established and maintained.

In order to enable controlled environmental conditions to be established in the climate zone or zones, the insect rearing farm further comprises an air conditioning zone Z4. The air conditioning zone in particular makes it possible for a large amount of air provided for the climate zone Z1, Z2, to be regulated to the desired temperature. The temperature regulation generally concerns the cooling of air. As a matter of fact, the rearing of millions of insects produces a large amount of heat, such that maintaining the climate zone at the desired target temperature basically consists of renewing the air present in the farm by providing fresh air.

The cooling of air may be obtained, in the air conditioning zone Z4, by an air conditioning system able to comprise various air cooling devices. Among these devices, the air conditioning system may comprise for example one or more air cooling towers. The air conditioning system may comprise one or more cooling units, for example one or more cooling units of centrifuge type. The air conditioning zone Z4 has the particularity of being able to generate two air streams concomitantly at different temperatures.

When the temperature outside the climate zone (typically outside the farm) is sufficiently low, that is to say substantially less than the temperature targeted in the farm, the cooling of the air may be obtained or complemented by the intake into the farm of air coming from the outside. For this, air extractors evacuate air from the climate zone to the outside, which is compensated for by the inlet of (fresh) air from the outside into the climate zone.

According to the invention, the thermal regulation of the climate zones, or of the silos of the climate zones, is carried out by delivering thereto two air streams at different temperatures. For example, for a given target temperature, also called setpoint temperature, of the order of 25° C. in the climate zone or the silo the air conditioning system may generate a first air stream at a temperature T1 and a second stream of air at a second temperature T2. For example, the first temperature T1 may be of the order of 14° C. For example, the second temperature T2 may be of the order of 8° C.

Thus, the temperature control may implement two air streams at temperatures less than the setpoint temperature. For example, the air stream at the first temperature can make it possible to drop the temperature in the climate zone Z1, Z2 partly, and mostly renew the air of the climate zone. Furthermore, the air stream at the first temperature can make it possible to mobilize a large amount of air in the climate zone Z1, Z2 and if required impart to it a swirl in order to promote the churning and mixing of the air in the said climate zone. The air at the second temperature can enable the temperature to be controlled quickly to attain the target temperature. The air at the second temperature can for example enable provision of a large amount of cold to the climate zone, which will be rapidly and homogenously mixed with the stream of air at the first temperature.

In order to supply each climate zone Z1, Z2, with air, the insect rearing farm comprises at least two sets of ducts. A first set of ducts C1 enables the air at the first temperature T1 to be transported from the air conditioning zone Z4 to each climate zone Z1, Z2. A second set of ducts C2 enables the air at the second temperature T2 to be transported to each climate zone Z1 Z2.

In the farm example represented here, a third set of ducts C3 enables a return of air from the climate zones Z1, Z2 to the air conditioning zone Z4.

The return of air towards the air conditioning zone Z4 in particular enables the recovery in the farm of air at a temperature close to the target or setpoint temperature, in order to generate in controlled manner the streams of air at the first temperature T1 and at the second temperature T2. The advantage in terms of power to implement for the cooling of the air is also high when the ambient temperature (outside the farm) is greater than the temperature of the air coming back to air conditioning zone Z4 by the third set of ducts C3.

Nevertheless, all or some of the air extracted from the climate zone Z1, Z2, may be so extracted by means of conventional extractors, for example situated at the location of the roof of the farm (where the hottest air can accumulate).

In order to ensure homogenous distribution of the air provided by the first set of ducts and by the second set of ducts into the climate zone, various configurations are possible. Three general configurations are described below, it being possible to envision numerous variants in each of these configurations.

A first configuration is in particular described with reference to FIGS. 3 to 9. A second configuration is described with reference to FIGS. 10 to 14.

In the first and in the second configuration, the first set of ducts and the second set of ducts comprise air distribution flues in the climate zone or zones. A distribution flue is a duct, straight or curved, comprising nozzles enabling the ejection of gas (typically air) from inside the flue to the outside. The nozzles may in particular have an orientation perpendicular to the direction of extension of the distribution flue. The term radial direction of distribution is used when the flue has a circular section, which is generally the case.

The nozzles may consist of simple apertures of specific caliber, provided in the wall of the distribution flue.

A distribution flue enables the air introduced into the climate zone Z1, Z2 to be spread. The first configuration and the second configuration of climate zone have different arrangements of the air distribution flues, making it possible to obtain a sufficiently homogenous temperature in the climate zone, and satisfactory renewal of the air.

The first general configuration, shown in FIG. 3, is based on a vertical distribution of the air distribution flues in the climate zone. FIGS. 3 to 9 more particularly show the first climate zone Z1 of a farm in accordance with FIG. 1, organized according to this first general configuration.

According to this first configuration, the ventilation system implemented thus comprises air transport ducts 4 in the upper part (for example running under the ceiling) of the climate zone Z1. The air transport ducts 4 belong respectively to the first set of ducts C1, to the second set of ducts C2, or to the third set of ducts C3.

Air distribution flues 5 extend vertically in the climate zone Z1. Each air distribution flue 5 belongs either to the first duct set C1 or to the second duct set C2, and thus enables the introduction of air into the farm either at the first temperature T1, or at the second temperature T2.

Optionally, air extraction flues 6, connected to the third set of ducts C3, may be provided. The air extraction flues 6 then advantageously have a vertical disposition similar to that of the air distribution flues 5, in this first climate zone configuration.

This ventilation system configuration is particularly suited to a climate zone which comprises parallel racks 7 provided to receive containers of insects (for example grouped into palletized sets such as that of FIG. 2).

The parallel racks 7 form parallel aisles 8 between them. In this first configuration is it possible to distinguish two types of aisles 8, according to their functional attribution. Some aisles, referred to as ventilation aisles 9 comprise distribution flues 5, and, if required, air extraction flues 6. Thus, the air transport ducts 4 extend advantageously above those aisles allocated to the ventilation system. The distribution flues 5 thus supply conditioned air to the racks 7 that are adjacent to the aisle in which they are disposed. Some aisles referred to as handling aisles 10 are provided for the movements of the rearing containers in the climate zone, as well as for their entry into the climate zone and for their exit from the climate zone (for example for carrying out a rearing operation in the third zone Z3 of the farm).

When the ventilation aisles have no air extraction flues, the extraction of air may be carried out at the ends of the aisles, or at one of the ends of the aisles in order to define a stream of air oriented within the aisles, by conventional extractors and/or by air extraction means connected to the third set of ducts C3. Alternatively, the extraction of air may be carried out on any side of the farm, according to the desired orientation of the stream of air. This disposition may also be implemented in addition to air extraction flues.

The movements of the containers, individually or in the form of basic rearing units BU, may be carried out using various systems. In particular, a storage and retrieval machine may be provided, which is able to move along—or between—the racks of the handling aisles 10. The storage and retrieval machine is for example configured to move the basic rearing units BU to or from an interface with the third zone Z3. This interface may comprise a belt conveyer. Other handling systems or more generally transport systems may be envisioned for the recovery of the rearing containers from the racks (or the installation of the rearing containers in the racks 7) and their movement. Robots, automatons, or autonomous transport vehicles may be employed, possibly with suitable elevators enabling said robots, automatons, or autonomous vehicles to be moved between the vertical levels of the racks 7.

The handling aisles 10 are thus provided with devices comprising a fixed structure for the transport of the basic units BU (storage and retrieval machine, elevator) or are left substantially free of any obstacle to facilitate the movement of the autonomous means (robots, automatons, or autonomous vehicles).

FIG. 4 illustrates, in a plan view from above, a climate zone in accordance with the general configuration of FIG. 3. In each ventilation aisle 9, flues 51 for distributing air at the first temperature are disposed alternately with flues 52 for distributing air at the second temperature.

This alternation is directed to ensuring good homogeneity in temperature in the climate zone. The alternation of the air distribution flues may consist of the following sequence: a flue 51 for distributing air at the first temperature, followed by a flue 52 for distributing air at the second temperature, followed by a flue 51 for distributing air at the first temperature, etc. Nevertheless, other sequences of alternation may be envisioned, in order to ensure optimal homogeneity in air temperature. Similarly, air extraction flues 6 may be disposed between the air distribution flues, preferably in a regular sequence. The distribution between the air distribution flues and the air extraction flues participates in establishing a stream enabling homogeneity in temperature and the renewal of the air in the farm. This stream is optionally optimized by a partial partitioning between the air distribution flues, of which an example is described below with reference to FIG. 5.

In the example represented here, the following sequence is provided along each ventilation aisle 9: an air extraction flue 6, followed by a flue 51 for distributing air at the first temperature, followed by a flue 52 for distributing air at the second temperature, followed by a flue 51 for distributing air at the first temperature, followed by a flue 52 for distributing air at the second temperature, followed by a flue 51 for distributing air at the first temperature, followed by an air extraction flue 6, followed if required by a new identical sequence (i.e. beginning with a new air extraction flue 6), etc.

In FIG. 4, arrows leaving the distribution flues 51, 52 illustrate the main direction of introduction of air from said distribution flues. In similar manner, arrows directed towards the extraction flues 6 illustrate the direction in which the air is sucked into said air extraction flues 6.

Thus, the flues 51 for distributing air at the first temperature tend to blow the air towards the racks 7, in order to ensure good renewal of the air in the rearing containers. The flues 52 for distributing air at the second temperature tend to blow the air at the second temperature in the direction of the ventilation aisle 9, towards the flues 51 for distributing air at the first temperature, in order for the air at the second temperature to mingle with the air at the first temperature before a relatively homogenous stream of air reaches the rearing containers.

The air extraction flues 6 suck the air from the racks 7, in order for a stream for renewal of the air is properly established in said racks.

FIG. 4 is a two-dimensional plan. Nevertheless, identical streams are provided over the whole height of the climate zone, or, at least, at several vertical levels of the climate zone, so as to provide a substantially identical and acceptable temperature over the whole height of the climate zone.

FIG. 5 illustrates this aspect. In particular, FIG. 5 shows a three-dimensional diagrammatic view of a rack 7 and the distribution and extraction flues adjacent to that rack 7.

Three planes P1, P2, P3 of air streams are shown, by way of illustration of air stream establishment not only in the lower part of the climate zone, typically close to the ground (at the level of the first plane P1), but also in an intermediate part of the climate zone (at the level of the plane P2) and in the upper part of the climate zone, typically close to a ceiling (at the level of the plane P3).

FIG. 5 also illustrates the possibility of putting in place partitions 11 making it possible to deviate the air streams in order to guide said streams of air within the climate zone and improve the homogeneity of temperature of the air reaching the racks 7 and the rearing containers.

Typically, partitions 11 may be disposed facing opposite outlet nozzles of the air distribution flues 5. As concerns the flues 51 for distributing air at the first temperature, this in particular makes it possible to avoid the entry into the racks of a direct stream of air from said flues 51 for distributing air at the first temperature. Such a direct stream of air could have too high a speed, and moreover, be detrimental to the proper mixing of the air at the first temperature T1 with the air at the second temperature T2.

Furthermore, partitions 11 may be provided between the air distribution flues 5 and the extraction flues 6, in order to limit the proportion of air introduced into the climate zone which would not participate in renewing the air in the rearing containers present in the racks 7.

FIG. 6 illustrates, in a plan view from above, a climate zone in accordance with the general configuration of FIG. 3, according to a second example embodiment. Just as in the embodiment of FIG. 4, in each ventilation aisle 9, flues 51 for distributing air at the first temperature are disposed alternately with flues 52 for distributing air at the second temperature.

In particular, in the example represented here, the following sequence is provided along each ventilation aisle 9: a flue 51 for distributing air at the first temperature is followed by a flue 52 for distributing air at the second temperature, followed by a flue 51 for distributing air at the first temperature, followed by a flue 52 for distributing air at the second temperature, etc.

In this configuration, the extraction of air is carried out at the location of one or more sides of the farm. In this case, air return vents 62 are formed on a first face of the climate zone, advantageously located in the vicinity or towards the air conditioning zone Z4. In particular, the air return vents 62 are advantageously located at an end of the aisles (in particular of the ventilation aisles 9) of the farm. The air return vents supply the third duct set C3, enabling a return of air from the climate zone to the air conditioning zone. The air coming from the third set of ducts is thus again temperature-regulated i.e. fully or partly at the first temperature T1 or at the second temperature T2.

Air extractors 63 are located on one or more sides of the climate zone. The air extractors 63 are advantageously located in an upper part of the climate zone, there where the hottest air tends to be located. The air extractors 63 enable the hot air present in the farm to be extracted to the outside, such that the air thus extracted is replaced by air coming from the outside. The renewal of the air may thus be performed via the introduction of air coming from the first and from the second set of ducts, or possibly via openings of the climate zone. When the outside air is at a substantially lower temperature than the temperature aimed for in the farm, this provision of outside air enables cooling in the climate zone without employing means for lowering the temperature of the air (air-conditioning systems of equivalent), such that only the energy cost of this cooling corresponds to the energy required for the extraction of air from the climate zone by the air extractors 63. This can thus be referred to as “freecooling”.

In a view similar to that of FIG. 6, FIG. 7 shows a variant of the embodiment of FIG. 6 making it possible to optimize the homogeneity of the temperature of the air in the climate zone.

According to the configuration shown in FIG. 7, the sequence provided along each ventilation aisle 9 is identical to that of the configuration of FIG. 6: a flue 51 for distributing air at the first temperature is followed by a flue 52 for distributing air at the second temperature, followed by a flue 51 for distributing air at the first temperature, followed by a flue 52 for distributing air at the second temperature, etc. Nevertheless, the air distribution flues, respectively at the first temperature and at the second temperature, are disposed in staggered arrangement between two consecutive ventilation aisles. Thus, in the transverse direction (considering that the aisles define the longitudinal direction), each flue 51 for distributing air at the first temperature is surrounded by flues 52 for distributing air at the second temperature, and each flue 52 for distributing air at the second temperature is surrounded by flues 51 for distributing air at the first temperature, except of course for the flues located in the aisles at the edge of the climate zone.

Similarly, according to a variant not shown, a different offset could be applied between the flues of two successive ventilation aisles (for example an offset by half the longitudinal distance between two distribution flues).

Furthermore, as the air extractors are located in the upper part of the farm, they may be installed at tower tops enabling the structure of the farm to be reinforced. In particular, the air extractors 63 may be installed at the top of towers 64 juxtaposed against a wall of the farm, such that not only do the air extractors 63 not constitute a load to bear for the structure of the farm, but the towers which support them can reinforce said structure of the farm. Such a configuration is represented in FIG. 8.

In the configuration example shown in FIG. 3, the air distribution flues are supplied by sets of ducts extending under the ceiling of the climate zone. Similarly, the air recovery flues are connected to the third set of ducts which also extend in the upper part of the farm.

Other configurations are nevertheless possible. FIG. 9 shows an alternative configuration example in a two-dimensional plan of the climate zone. According to this configuration, the racks are all disposed as in the configuration described with reference to FIGS. 3 to 8. In particular, the parallel racks 7 form between them parallel aisles 8, comprising ventilation aisles 9 and handling aisles 10 (typically one aisle in every two is a ventilation aisle and one in every two is a handling aisle).

A storage and retrieval machine or any other suitable handling means (in particular robots, automatons, or autonomous transport vehicles) may be provided in each handling aisle 10.

In contrast to the configuration of FIG. 3, one of the two sets of ducts enabling the introduction of air into the climate zone, in this case the second set of ducts C2 supplying the flues 52 for distributing air at the second temperature, extend in the lower part of the climate zone. In the example shown, the first duct set C1 supplying the flues 51 for distributing air at the first temperature extend in the upper part of the climate zone.

The fact that a set of ducts is located in the lower part makes it possible to limit the mass of infrastructures to be borne by the structure of the climate zone, in the upper part thereof. This also makes it possible to limit the total length of ducts in the farm, which is advantageous economically and in terms of reliability.

Of course, the inverse configuration to that presented in FIG. 9 may be envisioned, that is to say with the first set of ducts supplying the flues for distributing air at the first temperature extending in the lower part and the second set of ducts supplying the flues for distributing air at the second temperature extending in the upper part. In general terms, the first set of ducts, the second set of ducts, and optionally the third set of ducts (for the extraction of air) may extend in the lower part of the climate zone.

In the upper part or in the lower part of the farm, the set of ducts may comprise or be constituted by an air collector or plenum.

In the air distribution flues the supply by which is carried out from the bottom, the air stream rises, while in the air distribution flues the supply by which is carried out from the top, the air stream falls. Nevertheless, in either case, the air ejection nozzles of each flue are advantageously configured (in number, distribution, shape, cross-section) in order for the ejected stream of air to be substantially homogenous over the whole height of the racks 7 of the climate zone.

In general terms, it is preferable for the air stream reaching and passing through the rearing containers to have a low speed, such that this air stream is not liable to raise rearing material present in the containers.

In all the configurations employing vertical air distribution flues, such as those described above, the distribution of the air is advantageously distributed over the whole height over which insect rearing containers are present. In particular, the distribution of air is carried out up to approximately 50 cm below the lowest container (typically the crate). The lowest rearing container may be located at a certain height in relation to the floor of the farm, for example at approximately 1.5 meter from the floor, which makes it possible to receive a certain number of technical systems (storage and retrieval machine mechanisms, sets of ducts as described above, etc.) and also enables easy cleaning of the farm, for example using cleaning robots.

The second general configuration, is based on a horizontal distribution of the air distribution flues in the climate zone. Typically, the air distribution flues are disposed above the racks 7. FIGS. 10 to 15 more particularly show the first climate zone Z1 of a farm in accordance with FIG. 1, organized according to this second general configuration.

FIG. 10 shows a first variant of this second configuration, in a view in three dimensions, while FIG. 11 shows the variant of FIG. 10 in a two dimensional plan.

According to the second configuration, the racks 7 are organized into one or more vertically superposed strata, each comprising, in a same horizontal plane, several parallel rows 71, 72, 73. FIGS. 10 and 11 show a climate zone comprising three strata, namely a lower stratum S1, an intermediate stratum S2, and an upper stratum S3.

In the variant of the second configuration shown in FIGS. 10 and 11, a flue 51 for distributing air at the first temperature and a flue 52 for distributing air at the second temperature are disposed above each row 71,72,73. An air extraction flue 6 is disposed under each row. This configuration ensures there is an air stream through the racks in order to renew the air around the basic units (or unitary rearing containers) contained in said racks.

In order to provide a homogenous temperature in each of the strata, and in particular avoid the upper stratum S3 having a temperature substantially higher than the strata below it, the strata may be separated from each other by a thermally insulating floor. Furthermore, in this configuration, it is possible to ensure a temperature that is close between the different strata by commanding different rates of introduction of air at the first temperature and the second temperature in each of the strata S1, S2, S3. Typically, as the air at the first temperature is less cold than the air at the second temperature, it can be provided to distribute an amount of air at the second temperature (in proportion to the air at the first temperature) that increases with greater height of the stratum considered.

In the second general configuration, whether the climate zone is organized according to the first variant of FIGS. 10 and 11, according to the second variant of FIGS. 12 and 13, or according to another alternative variant, said climate zone can advantageously comprise parallel aisles configured for the passage of the rearing containers in the climate zone as well as for the entry of the rearing containers into the climate zone and their exit from the climate zone. These movements of the rearing containers, which may be grouped if required into basic units, may be made by means of various devices among which are a storage and retrieval machine, a belt conveyor, a robot, an automaton, or an autonomous vehicle.

According to the variant of the second configuration presented in FIGS. 12 and 13, no air extraction flue is provided beneath each row. The extraction of air may be made at the ends of the aisles, by conventional extractors and/or by air extraction means connected to the third set of ducts C3. An extraction of air at one of the ends of the aisles furthermore enables the creation of an air stream that is regular and oriented within the climate zone, according to the orientation of the aisles. Alternatively, the extraction of air may be carried out on any side of the climate zone, according to the desired orientation of the stream of air. Furthermore, in the variant shown, the climate zone is organized into groups of racks. Each group of racks is formed from an aisle, and racks located on either side of the aisle, that is to say the racks which are directly adjacent to it. FIGS. 12 and 13 thus each represent a single group of racks. A strata of a climate zone organized in the second configuration generally comprises a plurality of groups of adjacent racks. Optionally, for example to allocate two groups of racks to different silos, said groups of adjacent racks may be separated by a thermally insulating wall.

In the example shown, in a group of racks, above each rack 7 there is extends a flue 51 for distributing air at the first temperature and a flue 52 for distributing air at the second temperature, and above each aisle 8 there extends a flue 51 for distributing air at the first temperature.

The racks of the stratum shown have two layers, that is to say they are configured to store basic units BU on two levels.

The flues 52 for distributing air at the second temperature are advantageously located above the flues 51 for distributing air at the first temperature. This configuration makes it possible to dispose the flues 52 for distributing air at the second temperature (the second temperature T2 being colder than the first temperature T1) furthest from the racks 7 and from the rearing containers, which avoids a provision of air that is too cold in the rearing containers (the air at the second temperature T2 will necessarily be mingled with less cold air, typically at the first temperature T1, before arriving in the vicinity of the rearing containers).

Partitions 11 may be disposed between the air distribution flues so as to deviate the air streams coming out of the distribution flues to guide the air stream into the climate zone and improve the temperature homogeneity of the air reaching the racks 7 and the rearing containers they contain.

In particular, a partition 11 may be disposed facing opposite the nozzles of each of the air distribution flues, such that the air stream coming from each flue impinges on the partition respectively positioned opposite said flue. By way of illustration, in FIG. 13, arrows leaving the distribution flues 51, 52 illustrate the main direction of introduction of air from said distribution flues. The flues 51 for distributing air at the first temperature and the flues 52 for distributing air at the second temperature located directly above the racks 7 blow substantially horizontally, towards the center of the aisle 8 separating the racks, so as to create a direct stream of air between said flues and the racks. The flue for distributing air at the first temperature 51 located above the aisle 8 blows downwards, towards a partition 11 which promotes the mixing of the air coming from the different air distribution flues, and avoids creating a direct stream towards the bottom of the aisle 8, the zones located at the top (upper level of the racks 7) being the most difficult to cool, the hot air having a tendency to accumulate there.

FIG. 14 shows, in a two-dimensional plan, a third general configuration example of a climate zone of an insect rearing farm. According to this configuration, the racks are all disposed in the configuration described with reference to FIGS. 3 to 9. In particular, the parallel racks 7 form between them parallel aisles 8, comprising ventilation aisles 9 and handling aisles 10 (typically one aisle in every two is a ventilation aisle and one in every two is a handling aisle).

A storage and retrieval machine or any other suitable handling means (in particular robots, automatons, or autonomous transport vehicles) may be provided in each handling aisle 10.

A flue 51 for distributing air at the first temperature extends above each ventilation aisle 9. The air ejection nozzles of each flue 51 for distributing air at the first temperature are oriented towards the ground of each climate zone and of the corresponding ventilation aisle 9. Thus, the flues 51 for distributing air at the first temperature provide most of the supply of air into the climate zone and of the air stream therein. Flues 52 for distributing air at the second temperature extend between the flues 51 for distributing air at the first temperature. For example, one or two flues 52 for distributing air at the second temperature may be provided between two successive flues for distributing air at the first temperature 51.

The flues 52 for distributing air at the second temperature comprise distribution nozzles oriented towards the adjacent flue or flues for distributing air at the first temperature. The air at the second temperature T2 is thus mingled with the air at the first temperature T1 before reaching the racks 7, and is driven by the main air stream coming from the flues 51 for distributing air at the first temperature.

FIG. 15 shows a diagrammatic view in three dimensions of a variant of the climate zone of FIG. 14.

In the embodiment shown here, the extraction of the air is carried out at the ends of the aisles 8, by air extractors. The extractors are advantageously positioned in the top part of the handling aisles 10. In cooperation with the main stream of air distribution in the climate zone in the ventilation aisles towards the ground, such an extraction organizes a general air stream in the ventilation zone which passes through the racks 7, at all the levels of said racks 7. This enables a renewal of the air in all the racks and good homogeneity of the air (in temperature, humidity and CO₂ content) in the whole climate zone.

The configuration of FIG. 15 also has the particularity (optional in this configuration and moreover applicable to all the configurations of the invention, in particular the detailed configurations described above, and as shown in FIG. 7) that the air conditioning zone is directly adjacent to the climate zone. In particular, the air conditioning zone Z4 is arranged here above the climate zone. The climate zone Z4 in particular comprises several air processing stations 61. Each air processing station 61 makes it possible to bring air to the first temperature T1 or to the second temperature T2.

Other dispositions of the air conditioning zone Z4 may be envisioned, in particular on one side, on the floor, on the ceiling or against a wall of the climate zone. The insect rearing farm may also comprise several air conditioning zones, which may for example each comprise one or more air processing stations 61.

Thus, in the three configurations envisioned that are described above, free spaces are provided between the air distribution flues and the racks to enable homogenization of the air temperature. These spaces are located either in the ventilation aisles 9 of the first general configuration illustrated in FIGS. 3 to 9, or above the racks in the second general configuration illustrated in FIGS. 10 to 13, or above and between the racks as in the third general configuration illustrated in FIGS. 14 and 15. The walls 11 provided facing opposite the air ejection nozzles of the air distribution flues respectively at the first temperature and at the second temperature furthermore promote the stirring of the air introduced into the climate zone respectively the first temperature and at the second temperature.

The control of the temperature in the climate zone may be carried out by regulation of the amount of air respectively introduced at the first temperature and at the second temperature according to the difference between a setpoint temperature and a temperature measured at one or more points of the climate zone. If different setpoint temperatures are desired (for example if the climate zone is divided into several silos, or if several temperature sensors indicate that there is a large difference in temperature between different points of the climate zone) the amounts and proportions of air at the first temperature and at the second temperature may be regulated, by regulating valves, in different branches of the first set of ducts and in different branches of the second set of ducts. In the insect rearing farm example shown in FIG. 1, the first set of ducts C1 has a first branch B1 for introduction of air into the first climate zone Z1, and a second branch B2 for introduction of air into the second climate zone Z2. Regulating valves V1, V2, enable the apportionment of the air respectively introduced into the first climate zone Z1 and into the second climate zone Z2.

A similar configuration may be provided for the second set of ducts. In the same way, inside a same climate zone, each set of ducts may have several branches in which the throughput of air can be regulated independently.

The humidity level constitutes, as indicated above, another environmental parameter of which the control and command are important to promote the growth of the insects and limit the risk of developing certain diseases. The air conditioning system present in the air conditioning zone Z4 is advantageously configured to regulate the level of humidity of the air at the first temperature and/or the level of humidity of the air at the second temperature. If only one of the humidity levels of the air at the first temperature and of the air at the second temperature can be regulated, it may be adapted according to the level of humidity in the air at the other temperature, and according to the ratio between the air introduced at the first temperature and the air introduced at the second temperature. Furthermore, each climate zone may comprise additional air humidifying devices (for example misters) making it possible to correct the level of humidity, in order to attain a target humidity level. The air stream generated by the introduction and the extraction of air to and from the climate zone considered, and the kinematics of the air described with reference to the control of the temperature in the climate zone, enable good homogeneity of the humidity level in the air of the climate zone.

Lastly, the level of carbon dioxide also constitutes an environmental parameter of which the control is important. Maintaining a level of carbon dioxide at an acceptable level (under a predefined limit) is obtained by sufficient renewal of the air. To that end, a minimum rate of air renewal may be set. The renewal of the air is provided by concomitant introduction and extraction of a sufficient amount of air. It may thus be necessary, according to the level of carbon dioxide in the climate zone, to introduce air that is less cold in the climate zone, but in a larger amount. For example the amount of air introduced at the second temperature T2 (which may be expected to be cooler than the air at the first temperature T1) may be limited whereas the amount of air introduced at the first temperature is increased.

The invention thus developed enables effective regulation of the environmental parameters in an insect rearing farm, in particular in the context of farming at industrial scale. This regulation, in particular of temperature, is obtained by introducing air at two different temperatures in a same climate zone. Thus, the air streams can have roles that are mainly different. For example, the air introduced at a first temperature can participate in the cooling of the air in the climate zone, but also, in cooperation with air extraction means, generate most of the air stream in the climate zone. The stream generated has a function of renewal of the air in the climate zone and a function of homogenization of the air, whether in temperature, humidity or level of carbon dioxide. The air introduced at a second temperature, generally lower than the first temperature, enables fast correction of the temperature in the climate zone. Thus, the throughput of air introduced into the climate zone at the first temperature may typically (and according to the cooling needs) be two to four times greater than the throughput of air introduced into the climate zone at the second temperature.

An effective control of the environmental parameters in climate zones of large size (particularly for example several hundreds of square meters, with a height under the roof of several meters) makes it possible to envision insects farms at the industrial scale, with a maximized yield and good conditions for life and growth for the insects of the farm.

1. An insect rearing farm comprising a climate zone (Z1, Z2) which comprises a set of racks (7) for the storage of the insects in rearing containers (1, 2) and an air conditioning zone (Z4) comprising an air conditioning system configured for regulating the air to a first temperature (T1),
the farm comprising a first set of ducts (C1) configured to transport the air at the first temperature (T1) from the air conditioning zone (Z4) to the climate zone (Z1, Z2) and to deliver said air at the first temperature (T1) into said climate zone (Z1, Z2).
characterized in that the air conditioning system is furthermore configured for regulating the air temperature to a second temperature (T2) conjointly with the temperature regulation of air to the first temperature (T1), and in that the farm comprises a second set of ducts (C2) configured to transport the air at the second temperature (T2) from the air conditioning zone (Z4) to the climate zone (Z1, Z2) and to deliver said air at the second temperature (T2) into the climate zone (Z1, Z2), said air at the first temperature and said air at the second temperature mixing in said climate zone.

2. The insect rearing farm according to claim 1, wherein the farm comprises a device for extracting air which comprises a third set of ducts (C3) that are configured for the return of air, from the climate zone (Z1, Z2), into the air conditioning zone (Z4).

3. The insect rearing farm according to claim 1, wherein the first set of ducts (C1) comprises a plurality of flues (51) for distributing air at the first temperature, each formed from a duct comprising air ejection nozzles distributed along said flue (51) for distributing air at the first temperature, and in which the second set of ducts (C2) comprise a plurality of flues (52), for distributing air at the second temperature, each formed from a duct comprising air ejection nozzles distributed along said flue (52) for distributing air at the second temperature.

4. The insect rearing farm according to claim 3, wherein the racks (7) of the climate zone (Z1, Z2) are organized on opposite sides of parallel aisles (8) and wherein one aisle (8) in every two is a handling aisle (10) configured for the passage of the rearing containers (1, 2) in the climate zone (Z1, Z2) as well as for the entry of the rearing containers (1, 2) into the climate zone (Z1, Z2) and their exit from the climate zone (Z1, Z2), and one aisle (8) in every two is a ventilation aisle (9) which comprises a succession, in a predefined sequence, of flues (51) for distributing air at the first temperature and of flues (52) for distributing air at the second temperature, said flues (51, 52) for distributing air at the first temperature and at the second temperature extending substantially vertically between the racks (7).

5. The insect rearing farm according to claim 2, wherein the ventilation aisles (9) further comprise air extraction flues (6) of the air extraction device, said air extraction flues extending substantially vertically between the racks.

6. The insect rearing farm according to claim 5, wherein the flues for distributing (51, 52) and extracting (6) air may be arranged in each ventilation aisle (9) according to the following single or several times repeated sequence: air extraction flue (6), flue (51) for distributing air at the first temperature, flue (52) for distributing air at the second temperature, flue (51), for distributing air at the first temperature, flue (52) for distributing air at the second temperature.

7. The insect rearing farm according to claim 4, wherein the air distribution flues (51, 52) (6) are arranged in each ventilation aisle (9) in the following single or several times repeated sequence: flue (51) for distributing air at the first temperature, flue (52) for distributing air at the second temperature, flue (51), for distributing air at the first temperature, flue (52) for distributing air at the second temperature.

8. The insect rearing farm according to claim 2 further comprising air return vents (62) of the air extraction device, located at an end of said aisles (8).

9. The insect rearing farm according to claim 1, further comprising air extractors (63) configured to extract air from the climate zone to the outside of the farm.

10. The insect rearing farm according to claim 9, wherein the air extractors (63) are in the upper part of the climate zone, on towers (64) juxtaposed against a wall of the farm.

11. The insect rearing farm according to claim 3, wherein the flues (51) for distributing air at the first temperature and the flues (52) for distributing air at the second temperature are disposed above the racks (7).

12. The insect rearing farm according to claim 11, wherein the racks (7) are organized into one or more strata (S1, S2, S3) each comprising, in a same horizontal plane, several parallel rows (71, 72, 73), in which a flue (51) for distributing air at the first temperature and a flue (52) for distributing air at the second temperature are disposed above each row (71, 72, 73), and an air extraction flue (6) of the air extraction device is disposed under each row (71, 72, 73).

13. The insect rearing farm according to claim 12, wherein the racks (7) of the climatic zone (Z1, Z2) are organized, in one or more strata (51, S2, S3), on opposite sides of parallel aisles (8) configured for the passage of the rearing containers (1, 2) in the climate zone (Z1, Z2) as well as for the entry of the rearing containers (1, 2) into the climate zone (Z1, Z2) and their exit from the climate zone (Z1, Z2) and in which above each rack (7) there extend a flue (51) for distributing air at the first temperature and a flue (52) for distributing air at the second temperature, and above each aisle (8) there extends a flue (51) for distributing air at the first temperature.

14. The insect rearing farm according to claim 12, wherein the racks (7) of the climate zone are organized on opposite sides of parallel aisles (8) and wherein one aisle (8) in every two is a handling aisle (10) configured for the passage of the rearing containers (1, 2) in the climate zone (Z1, Z2) as well as for the entry of the rearing containers (1, 2) into the climate zone (Z1, Z2) and their exit from the climate zone (Z1, Z2), and one aisle (8) in every two is a ventilation aisle (9) above which extends a flue (51) for distributing air at the first temperature of which the air ejection nozzles are oriented towards a ground of the farm.

15. The insect rearing farm according to claim 14, wherein the air ejection nozzles of the flues (52) for distributing air at the second temperature are oriented towards a flue (51) for distributing air at the first temperature.

16. The insect rearing farm according to claim 3, wherein walls (11) are provided facing the air ejection nozzles of the air distribution flues (51, 52), so as to promote the mixing of the air introduced into the climate zone (Z1, Z2) respectively at the first temperature (T1) and at the second temperature (T2).

17. The insect rearing farm according to claim 1, wherein the air conditioning system furthermore enables the control of the humidity level of the air at the first temperature (T1) and/or of the air at the second temperature (T2).

18. The insect rearing farm according to claim 1, wherein the first temperature (T1) is greater than the second temperature (T2), the air conditioning system being configured to produce two to four times more air at the first temperature (T1) than air at the second temperature (T2).

19. The insect rearing farm according to claim 1, wherein the first set of ducts (C1) and the second set of ducts (C2) may each comprise branches (B1, B2) provided with control valves (V1, V2) making it possible to modulate the rate of flow of air into each of said branches.

20. A method for air conditioning in a climate zone of an insect rearing farm, the method comprising introducing air at a first temperature (T1) into the climate zone (Z1, Z2) conjointly with introducing air at a second temperature (T2) into the climate zone (Z1, Z2) and with extracting from said climate zone (Z1, Z2) a similar amount of air to the amount of air introduced, and controlling the amount of air respectively introduced at the first temperature (T1) and at the second temperature (T2) according to the difference between a temperature setpoint and a temperature measured at one or more points of the climate zone.

21. The method of air conditioning according to claim 20, wherein the first temperature (T1) is greater than the second temperature (T2), the first temperature (T1) and the second temperature (T2) both being less than the setpoint temperature.
